(19) United States
(12) Patent Application Publication
(10) Pub. No.: US 2010/0049223 A1
(43) Pub. Date: Feb. 25, 2010

(54) PROSTHESIS FOR ANASTOMOSIS

(76) Inventor: Luiz Gonzaga Granja Filho, Recife/PE (BR)

Correspondence Address: VOLPE AND KOENIG, P.C., UNITED PLAZA, SUITE 1600, 30 SOUTH 17TH STREET, PHILADELPHIA, PA, 19103, US

(21) Appl. No.: 12/303,537

(22) PCT Filed: Jun. 6, 2007
(86) PCT No.: PCT/BR07/00146

§ 371 Date: Jul. 8, 2009

(30) Foreign Application Priority Data
Jun. 6, 2006 (BR) PI 0602735-0

(51) Int. Cl. A61B 17/08

(52) U.S. Cl. 606/153

(57) ABSTRACT

A prosthetic device is provided with a double flange used for anastomosis of extremity with lateral, extremity with extremity and lateral with lateral without clamping and sutureless or with clamping and sutureless, in which a graft is inserted in at least one of the intraluminal parts of the tubular member of the prosthesis and is fixed to the internal flange of the prosthesis by a circular point or another method. The present invention describes different ways of fixing the flanges when they are in separate parts, making sure that there will be no protuberance of the anastomotic set in the lumen of the organ.

### FIELD OF THE INVENTION

The present invention refers in general to anastomotic devices and, more specifically, to a prosthetic device with double flange allowing anastomosis without clamping and suture, or with quick clamping and sutureless, the latter in organs with normal walls, from which a vascular graft or any other tubular one is inserted into the lumen of the prosthesis and removed by casing to cover part of the prosthesis, which will remain inside the graft (vein, artery or tissue) and is fixed to the internal flange of the prosthesis either by a circular stitch or another method. The flanges of the prosthesis may have a plurality of spaced openings on their periphery, allowing the prosthesis to be sutured in the tissue, vein, artery or any other organ outside the anastomosis, thus eliminating one of the leading causes of stenosis and anastomosis obstruction, which is the introduction of foreign bodies into the lumen, as a strange body-type reaction occurs, such as hyperplasia and the proliferation of the intima layer and cicatricial fibrosis, as well as the leading factor responsible for thromboembolisms, tissual lacerations and ischemia of the upstream organs, which is clamping, especially of friable and/or calcinated structures.

### DESCRIPTION OF THE PRIOR ART

A prior art presents several trials provide solutions for anastomotic devices projected to correct vascular abnormalities, which present the following typical features:

The North-American U.S. Pat. No. 3,254,650, of Jun. 7, 1966, describes a method and devices to execute anastomosis procedures by applying with adhesive two separated connectors in a body member and removing this body member portion contained among the connectors, joining the said connection devices for joining the remaining portions of the body member.

The U.S. Pat. No. 3,265,069, of Aug. 9, 1966, describes devices or instruments for use by surgeons in reunion of body ducts, which in the course of operations were separated. The instruments comprise a pair of elongated similar elements and articulatedly connected, in an intermediary manner, and with an support for finger retention in a distal end, comprising a generally cylindrical shape with a cylindrical channel that passes through it in the other distal end, in order to receive tubular body ducts kept by the instrument while the body ducts are reconnected.

U.S. Pat. No. 3,774,615, of Nov. 27, 1973, describes a device to connect the end of interrupted tubular organs without sewing, comprising a connecting ring on which the end of the interrupted organ are pulled, the ring is preferably locked up by a fixation resource. The ring and fixation resource are made of inert material, and preferably a hydrophile gel that can be dilated until its equilibrium or can be a hydrogel incompletely dilated, which is submitted to additional dilatation where it is applied. The connecting ring can be supplied with a groove and can be placed in a ring shaped fixation resource and kept there joining it to the fixation resource in the groove or simply kept by a screw. Two connection rings can also be used and kept joined by a coupling member.

The document U.S. Pat. No. 4,366,819, of Jan. 4, 1983, describes an anastomotic joint for surgery with a graft of coronary artery deviance comprising a mounting of four elements including a cylindrical tube with at least one locking indentation of ring flange in one influx end and a plurality of grooves of locking ring in a flow end; a ring flange with a central opening and a plurality of long and short spigots, the long spigots are engaged in the locking indentation, with a graft engaged among them; a fixation ring with a central opening and a plurality of spigots positioned around the opening; and a locking ring with a opening with a plurality of locking ring edges for engaging with the locking ring grooves. In surgical implants, an aortic wall with a hole engages between the ring flange and the fixation ring and is kept in this position by spigots of the fixation ring, and the four elements engage together forming an integral anastomotic joint. A first alternate modality includes an anastomotic joint of three elements with a combination of fixation ring and locking ring. A second alternate modality includes an anastomotic joint of four elements with a slightly jolted end in a influx end, exposing the graft material in the anastomotic “ostium”.

Other prior arts are equally mentioned, base don some information of “The Cardiothoracic Surgery Network”. The “Simmetry Aortic Connector System”, developed by St. Jude Medical, is a connector made with nitinol, selected by vein diameter with an adventitia removed to allow adjust of the connector and to prevent its displacement by the blood current. Then, the device may make an angle of 90° with the aorta. Among the disadvantages, there is the fact that it can be used only in extreme cases due to the difficult usage of this technique; it did not obtain a satisfactory result in many surgeries and it is being drowned out of market by the manufacturer; it is not applicable in calcified aorta; presents suture; presents contact with blood flow (foreign body); it does not widen the anastomosis area (restrictive anastomosis); performs only one anastomosis at a time; it is a product restrict to end-to-side anastomosis; a great mobilization of the venous graft occurs, damaging it, and can eventually form thrombus; there is a risk of perforation of the posterior wall of aorta; and the adventitia is removed (most resistant vascular layer).

Other known device is the PAS-Port™System, a device used in 3 steps, and the vein wall is mounted over the device and is manually reversed on it, by tool and adapted to aorta with a angle of 90°. The method alerts that the surgeon shall select with due care the point of aorta and the vein size. The device is made of stainless steel and is available in only one size that allows the use of veins with external diameter of 4 to 6 mm, aorta with an internal diameter of 18 mm. It is available in only one size, limiting its applicability. As disadvantages of this prior art, the device has contact with blood flow (foreign body); it does not widen the anastomosis area (restrictive anastomosis); it uses veins with external diameter of 4 to 6 mm and aorta with an internal diameter of 18 mm; it does not perform multiple nor visceral anastomosis; it performs just only end-to-side anastomosis; a great mobilization of the used biological graft occurs, damaging its inner layer, which generates the formation of thrombus; there is a big risk of kinking at the origin (angle of 90°) and risk of posterior wall perforation in the aorta at the moment the device is introduced under its light; the suture is substituted with disadvantages by stainless steel (9 pins, distant among them, maximizing the risk of bleeding).

Also as prior art, there is the CorLink Device, currently commercialized by Ethicon/Johnson & Johnson, that allows the creation of anastomosis between the ascending aorta and a saphenous vein segment. Aortic Anastomotic Device (AAD) is a self-expanded device with extra luminal nitinol constituted by a de um central cylinder with five interconnected elliptical arches and 2 groups of 5 pins in the end portion of the cylinder. The pins, after the eversion of venous walls in the device, fix the aggregate penetrating into the venous graft wall. A blade makes an opening in the wall of aorta and permits the coupling of AAD, which also fix the wall of aorta by pins. With this device: it poses a serious risk of bleeding, especially in friable aortas, thin, calcified or fibrous, restricting its applicability, also with risks, even in aortas with normal walls; in small gauge anastomosis, there is a risk of thrombosis, hyperplasia, intimal proliferation and fibrosis (reaction to foreign body type in origin of anastomosis) with consequent stenosis resulting in occlusion of anastomosis; sutures are used in some cases; there is cases of infarction caused by equipment; there is a recurring need of re-operations in patients; the device presents contact with blood flow (foreign body); it is not flexible; it does not multiple anastomosis; an inadequate mobilization of venous graft occurs, and can cause damage to its intimal layer, it could form thrombus; it is used only in extreme cases because it is a technique of complex usage; the suture is substituted by stainless steel in contact with blood flow.

Another known device is the St Jude Distal Connector that consists of a stainless steel clip mounted on a catheter, comprising a balloon for subsequent expansion and connector mounting. The catheter is introduced backward from the end, by doing a small hole in the anastomosis site, the clip fixes the vein in the hole, the catheter goes to coronary and releases the connector. The catheter is removed and a suture is done in side-to-side anastomosis. With St Jude Distal Connector, occurrence of leakage problems were detected in 20% of the used connectors; the use of a metallic clip requires due care for handling to avoid distortion in the anastomosis; late angiographies reveal smaller circular diameter of anastomosis made with o St. Jude Distal Connector, when compared to controls made with conventional suture; there is remarkably risk of bleeding and the graft is very mobilized, and lacerations can occur in its inner layer, allowing the formation of thrombus.

The HeartFlo™ is a multi-suture instrument for anastomosis with wires automatically applied in end-to-side and side-to-side anastomosis. The surgeon manually ties the suture wires (10 wires) and concludes the anastomosis similarly to the traditional process. Besides of being a product of complex handling, it makes suture in anastomosis (keeping the undesirable foreign body in the internal origin of the anastomosis) and is restricted to end-to-side and side-to-side anastomosis. There is also an excessive mobilization of graft, and can cause lesions in its intimal layer, which would be the inductor that forms the thrombus.

Another technique and known device is the Solem Graft connector, produced by the Swedish company Jomed. It is constituted by a stent made of nickel and titanium coated with polytetrafluorethylen used to connect the internal thoracic artery the left anterior descending coronary artery. The results has not been satisfactory, because it poses risk of bleeding; there is also an excessive mobilization of graft, probably damaging intimal layers, allowing the formation of thrombus; it is not flexible, by this fact, causes trauma to grafts; it does not make multiple anastomosis, at a single time; presents contact with blood flow (foreign body); and is frequent the need of-operations.

The Magnetic Vascular Positioner System is produced by Ventrica and comprises 4 magnetic rings and the anastomosis is processed by magnetic attraction of 4 ports. However, initial experimental results demonstrate leakage, also a undesired contact of materials with blood flow. On the other hand, it is necessary to be careful to avoid the capitation of excess of tissue among the magnets. With this system, there is also a need of suture in some cases; there is occurrence of infarction caused by equipment; and is frequent the need of-operations in patients; and also requires clamping.

Also, as a device known by the medical area, the Combined Anastomotic Device and Tissue Adhesive, developed by Grundeman & Borst group, combines micro mechanical technique with use of adhesive (glue). The use of this method can result in leakages and need traditional sutures; it is frequent the need of re-operation due to leakage/bleeding; and performs only one anastomosis at a time.

Finally, it is also experimentally practiced anastomosis assisted by laser, where the results are not different from conventional isolated sutures, because there is a need of suture in some cases; there is a risk of bleeding e leakage; and does not perform multiple anastomosis.

Even so divulged nowadays, anastomosis with clamper, by insecurity, and almost totality of surgeons perform conventional sutures throughout the route of anastomosis, with an intention of avoiding leakages and bleedings, it means the use of clampers just makes the procedure more expensive, once the conventional suture is also applied.

In short, the conventional anastomosis, with clamping and with suture, standardized in 1906 by Aléxis Carrel, remains the first choice for any type of anastomosis and organs to be anastomosed.

With an expectation of changing the current situation, the Brazilian patent no. PI 9706197-2, describes and claims a prosthesis for vascular anastomosis, or in any other organ or tissue, without the use of clamping and sutureless, solving, in an elegant and efficient manner, the limitations inherent to prosthesis of the above mentioned prior art, when used in vascular anastomosis performed, mainly in thin aortas, calcified and friable; or in any other application where a clamping of a vein or artery can pose excessive trauma for conditions of a given patient. The prosthesis that is subject of that request allows the embodiment of fast and safe anastomosis, without obstruction of vein or artery lumen of which anastomosis is made, also allows anastomosis in tissues, veins or arteries in bad conditions and never would accept a clamping used in conventional anastomosis. This is achieved by a generally cylindrical shaped prosthesis with a flange orthogonally extending from its external side wall, in a point in the prosthesis length between its ends; the referred flange has openings distributed around its surface. The description of the usage method and specific construction of the prosthesis is presented in the drawings of the descriptive report of that request, as well as the document C19706197-2, Certificate of Addition of the first.

Although these anastomotic devices can be presented as suited to the purposes for which they were projected, they are not so suited for the purposes of the present invention, as described herein below.

### SUMMARY OF THE PRESENT INVENTION

The present invention refers to the variations of the anastomotic devices currently known, so as to allow latero-lateral, termino-terminal and termino-lateral anastomoses without clamping and sutureless or, in normal wall organs or in which clamping does not represent risks or aggressions (intestines, etc.), with quick clamping sutureless where at least one vascular graft with expanded extremity or anastomotic trunk (formed by the union, by any method, of the extremities of two or more grafts forming a single mouth from which grafts individualize themselves), or any other, is inserted into the lumen of the prosthesis with double flange and turned over by casing to cover part of the prosthesis, being fixed to the internal flange. The prosthesis may also be in a single part or in more parts, each one of them including a flange made with equal or different materials. The flanges may or may not have openings, allowing them to be sewn onto the outer side of the tissue, vein, artery or tubular organ in order to eliminate contact of foreign bodies with the inside of the anastomosis. The prosthesis may also have varied dimensions and shapes in order to simultaneously receive varied sizes and types of grafts, as well as to be made in any biologically compatible material (tissues or polymers) or synthetic ones (stainless steel, titanium, nitinol, pyrolitic coal, silicone, Dacron, PTFE, Gor-Tex) or any biodegradable material.

One objective of the present invention is to bring forth an anastomotic device equipped with a tubular member and at least two flanges in a single or more than one piece of different materials, allowing the insertion of more than one graft of different types and calibers (such as, for instance, anastomotic trunk with autologous, homologous or heterologous biological grafts made with the same tissue or mixing them even with synthetics) in the same prosthesis.

Another objective of the present invention is to provide an anastomotic device that does not introduce any foreign bodies into the anastomosed grafts and the anastomosis area.

It is still the objective of the invention to allow an anastomosis without any protuberance caused by the sandwiching of the anastomosed vessel wall.

Additional objectives of the present invention and other modalities will come up as the description proceeds. Such modalities will be described in enough details in order to allow experts in the matter to implement the invention. Moreover, it must be understood that other modalities may be used and that structural changes may be carried out without distancing themselves from the scope of the invention. In the accompanying drawings, characters of similar reference name the same parts or similar ones throughout the several viewings.

Thus the following detailed description must not be taken in a limiting sense, and the scope of the present invention is better defined by the annexed claims.

### BRIEF DESCRIPTION OF THE DRAWINGS

For the invention to be more fully understood, now it will be described by means of an example in reference to the annexed drawings, of which:

FIG. 1 illustrates a first realization of the prosthesis with double flange, in a single piece, with internal flange in the intraluminal part smaller than the external flange.

FIG. 2 illustrates a second realization of the prosthesis with double flange in a single piece, with the internal flange in the intraluminal part smaller than the external flange; with the external flange including a triple head, with the possibility of being pleated or not.

FIG. 3 illustrates a third realization of the prosthesis with double flange in two parts; the internal part has a smaller diameter than the external part and is equipped with tabs on the surface of the small flange, and the external part has a larger flange with double holes through which the threads that will fix the two parts together will pass.

FIG. 4 illustrates a fourth realization of the prosthesis with double flange in two parts, the internal part having a smaller diameter than the external one and equipped with tabs on the surface of the small flange, and the external part having the larger flange with double holes through which the threads that will fix the two parts together will pass and including a triple head, which may be pleated or not.

FIG. 5A illustrates a fifth realization of the prosthesis with double flange in two parts, the internal part having a smaller diameter than the external one and equipped with tabs on the surface of the small flange, and the external part of the larger flange having two holes through which the threads that will fix the two parts together will pass, the external surface of the upper extremity of the tubular body of one part (small-size screw thread) and the internal surface of the lower extremity of the tubular body of the other part having screw threads for their interconnection (small-size screw threads, two or three small tabs).

FIG. 5B illustrates the fifth realization of the prosthesis with double flange, in which the upper flange has holes with female screw thread, obliquely to its thickness, to connect sticks or stems that will help in threading, considering that, differently from 5A, the internal and external screw threads occupy all extension of the parts' tubular members and not only their extremities, such as in 5A.

FIG. 5C illustrates the fifth realization of the prosthesis with double flange, in which the upper part includes only the flange, equipped with holes with female thread obliquely to its thickness in order to connect sticks or stems that will help thread the upper flange, fixing itself to the lower part with nippers especially created for that purpose, allowing threading even in hard-to-reach spots, similar to the threading of the parts of the prosthesis in FIG. 5B.

FIG. 6 illustrates a sixth realization of the prosthesis with double flange in two parts, the internal one with a diameter smaller than the external part and equipped with tabs on the surface of the small flange, and the external part equipped with the larger flange with double holes through which the threads that will fix the two parts together will pass and including a triple head, which may be pleated or not, and the external surface of the tubular body of one part and the internal surface of the tubular body of the other part having small-size screw threads in their extremities for their interconnection.

FIG. 7 illustrates a seventh realization of the prosthesis with double flange in a single piece, the intraluminal flange being rigid, with the possibility of being metallic or made of any biocompatible material, and the external flange, larger and flexible, with the possibility of being biological (autologous, homologous or heterologous), of synthetic tissue (Dacron, PTFE, silicone, Gor-Text) and non-metallic.

FIG. 8 illustrates an eighth realization of the prosthesis with double flange in two parts, the internal one having a smaller diameter than the external one and equipped with tabs on the surface of the small flange, and the external part having the larger, flexible flange, with the possibility of being in biological, synthetic and non-metallic material and including a triple head, pleated or not, with the external surface of the tubular body of one part and the internal surface of the tubular body of the other part having small screw threads for their interconnection.

FIG. 9 illustrates the ninth realization of the prosthesis with double flange, in two magnetized parts so they attract each other, with the external surface of the tubular body of one part and the internal surface of the tubular body of the other part still with the possibility of having small screw threads for their interconnection.

FIG. 10 illustrates the tenth realization of the prosthesis with double flange, and a spring between them acts as a tubular body which, with the help of stretching sticks, might be adjusted to any thickness of the tissue.

FIG. 11 illustrates the eleventh realization of the prosthesis with double flange and an elastic rubber or any flexible material between them acting as a tubular body which, with the help of stretching sticks, may be adjusted to any tissue thickness.

FIG. 12 illustrates a new modality of prosthesis with double flange, in two parts, the first flange being equal to the second flange, the first flange having the intraluminal part that will be coated by the graft and the second flange not having the intraluminal part.

FIGS. 13A-13F describe the realization stages of the anastomosis with the new modality of the prosthesis of FIG. 12.

FIGS. 14A-14C describe the realization stages of the anastomosis with the modality of the prosthesis of FIG. 12, the second flange being a graft of biological or synthetic tissue.

FIG. 15 illustrates a second realization of the modality of FIG. 12, the first flange having two, three or more pleated, flexible, biological or synthetic heads.

### DESCRIPTION OF THE FAVORITE MODALITIES

Now, regarding the drawings, in which similar reference characters show elements similar for all several viewings, the figures illustrate one of the realization forms of the present invention, in the form of prosthesis for anastomosis with double flange and different materials.

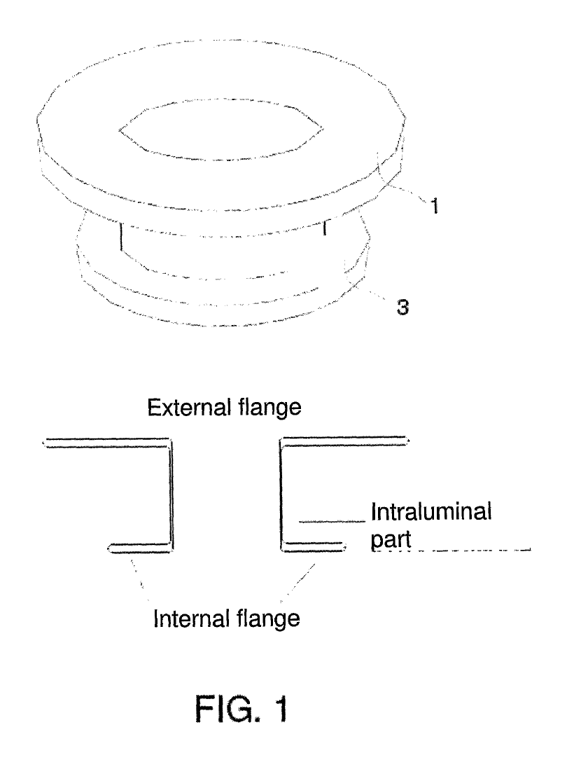

FIG. 1 presents a double-flange prosthesis, in a single piece, with external flange 1, with the possibility of having internal or external 2 holes and double holes or tabs to anchor the tabs of the threads so they will not obstruct the prosthesis lumen (may be applied only with suture in the pocket of the organ wall, incision of the wall, its introduction and ulterior thread tie-up with circumferential fixing) and internal flange 3 in the intraluminal, smaller part that is anchored into the internal surface of the organ in which it will be introduced (asymmetric spool).

In all prostheses having double, intraluminal flange, if it is wanted to recover the intraluminal part with any biocompatible tissue, preferably with the tissue from the patient him/herself (autologous), this will only be possible, without harm to the initial caliber of the graft(s), if an anastomotic trunk is made through the union, by any safe, efficient method (ex: seromuscular suture, preferably) of its ends, longitudinally opened at the wanted extension for the trunk. The trunk must have sufficient extension and diameter to pass through the lumen of the prosthesis, to be everted and to recover the intraluminal part with its flange. If only one graft is going to be used, its extremity that will cover the internal flange and the intraluminal part must be expanded to the necessary extension and diameter by being longitudinally incised on the correct extremity (care must be taken in grafts with valves, such as veins) and interposing, for instance, with seromuscular suture (the threads of which are not exteriorized in the lumen of the graft), any biological or synthetic tissue, preferably the tissue of the patient him/herself, in order to avoid the presence of a strange body in the site of the anastomosis. The advantage that this might present in regards to the internal, flange-free prostheses, whose single grafts might have their external diameters made compatible with the internal diameter of the prostheses and, thus, avoiding everting difficulties, is that in these prostheses with double flange, sandwiching the walls of the organ to which the prosthesis-graft set is being fixed, will make sure that no protuberance of the set will be in its lumen, preventing the presence of any foreign body, such as suture threads (if the points were transfixant from the organ wall) or that any wall tissue, such as calcification, atheromatose plaques, excess of internal layers, such as the intima of the vessels and mucosae, fibrosis spicule, etc., internally interpose themselves to the origin of the anastomosis, which are aspects of key importance for the long-term success of the procedure.

The prosthesis of FIG. 1 may have varied forms and dimensions of its flanges and intraluminal parts, depending on the characteristics of the walls of the organ where it will be fixed.

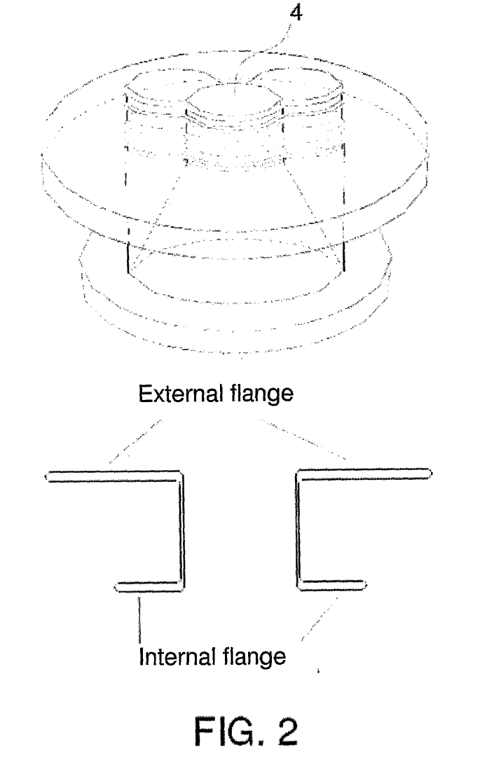

FIG. 2 represents the prosthesis in a single piece, triple head 4, pleated, movable, of the same or different material, to be externally coated by the grafts, which will be fixed with an external circular stitch or another efficient method. Internally, this prosthesis has a single mouth and conic form when individually heading to the exit, in order to take up the physiological, laminar flow in the case of vessels. It is clear that in this prosthesis there is no concern about recoating its intraluminal part by autologous tissue. The prosthesis may be internally coated by any tissue, including the autologous one, thus avoiding contact of the blood or fluid with the material the prosthesis is made of. In case it is not internally coated with autologous tissue, it must be as calibrous and short as possible, and can be round, oval or elliptical so as to avoid a lengthy contact of the blood or fluid with non-autologous material for, in this case, the flow will be higher. The heads 4 of the prosthesis in FIG. 2 are pleated and may be directed to any position and have a rigid extremity where grafts will be circumferentially and externally fixed. They may be of any biological or synthetic tissue. They may be fixed to the rest of the prosthesis by any safe method, such as circumferential ligature around small upper extensions of the prosthesis, glue, suture, etc.

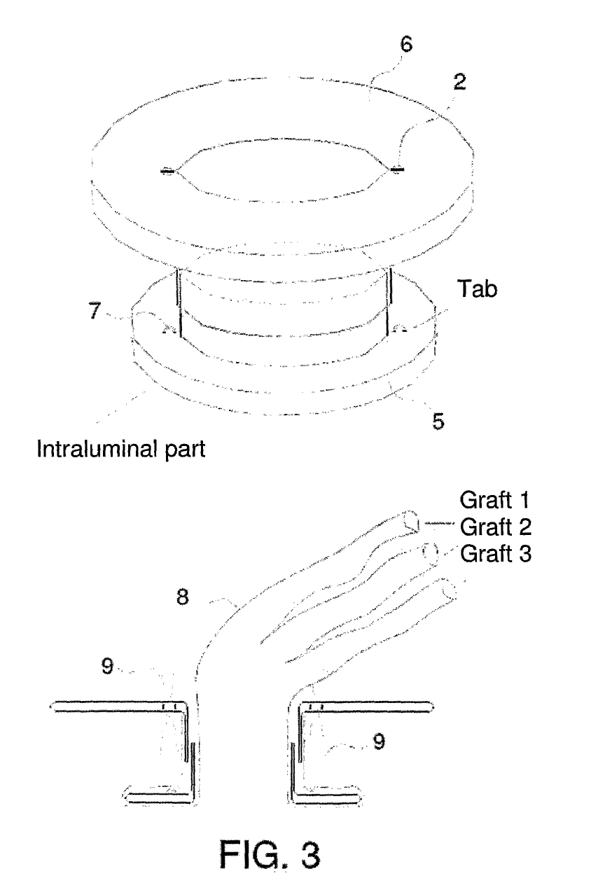

The prosthesis of FIG. 3 has two flanges and is made up by two pieces: internal 5 and external 6. The internal piece 5 has a flange in a smaller diameter with two small tabs 7, diametrically opposed, almost adjacent to their tubular member and situated on its upper surface. The external piece 6 has a bigger flange with double holes 2, diametrically opposed, coinciding with tabs 7 of the intraluminal flange. The external surface of the tubular member of the external part 6 has transversal grooves 12 for fixing of the everted graft or grafts or the anastomotic trunk. After the coating of the internal part 5 by the anastomotic trunk 8 (such as seen in the lower drawing of FIG. 3), of its flange and the tubular member of the external part 6, this one being fixed with an external circular stitch, two other threads 9 (which can be elastic threads or springs—both the tabs and the threads may be situated on the internal face of the external and internal parts) are passed by the small tabs 7 of the internal flange 5 heading upwards, trespassing the double holes 2 of the external flange 6. After the incision and introduction of the prosthesis-grafts set in the lumen of the organ, these threads 9 are drawn, causing a sandwiching of the organ wall, thus making sure that there will be no protuberance or hemostasia, besides fixing the parts 5,6 together. If wanted, it is possible to make a serous or seromuscular suture around the prosthesis for reinforcement of the hemostasia or to avoid leakage of any fluid, either blood or not.

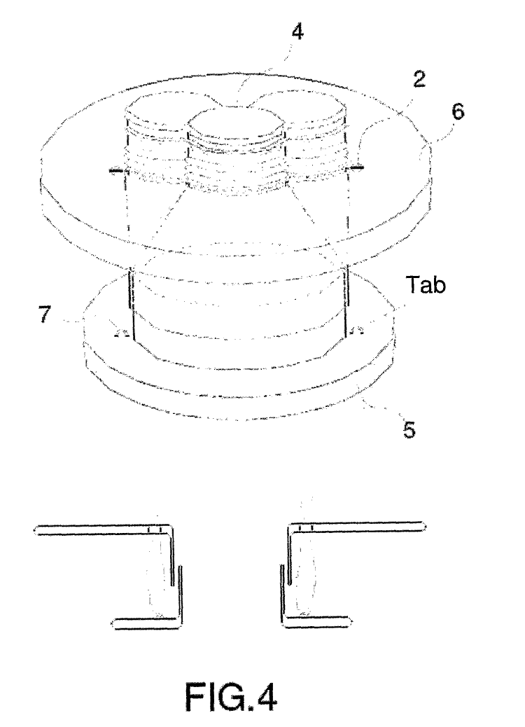

The prosthesis of FIG. 4 has equally two parts 5,6 (which will be united by inelastic or elastic thread, spring, etc.), external and internal, double holes 2 on the external flange 6, small tabs 7 diametrically opposed on the internal flange 5, which might also be situated on the internal surface of both parts. The external part 6 of the prosthesis of FIG. 4 has triple head 4, short, pleated, with rigid extremity, which will be externally and individually coated by grafts with external circular stitch. These heads 4 may be equally fixed to the other part of the prosthesis by any safe method such as suture, glue, circular ligature over the small upper extension of the other part of the prosthesis, etc. This prosthesis may also be internally coated by autologous tissue (for instance, free-dried autologous or micro fragments of the vascular intima or mucosae of the wanted organs, fixed onto an incompletely micro furrowed or vented internal surface in order to facilitate fixing with biological glue or another method) or by any other non-autologous tissue. It may not be coated and come straight from the factory ready to be applied. The technique for its use is similar to the one previously described for FIG. 3, and may have other technical realizations. The organ wall is incised at the center of the serous or seromuscular suture. The other possibility is total incision in pocket, previously made or not, by introducing the prosthesis-grafts set, with the threads unifying the parts of the prosthesis drawn and tied up and the organ wall being sandwiched. And, if the pocket suture is made, its legs are tied up, guaranteeing fixing of the prosthesis and hemostasia.

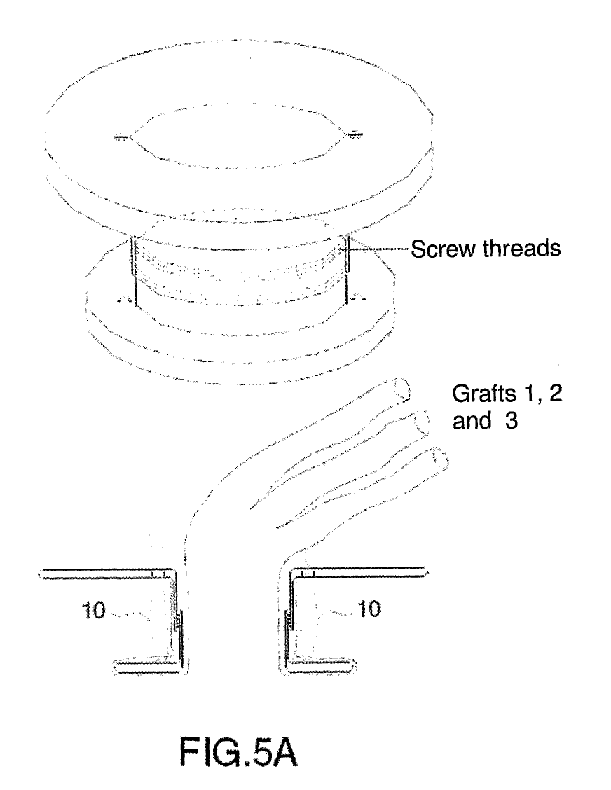

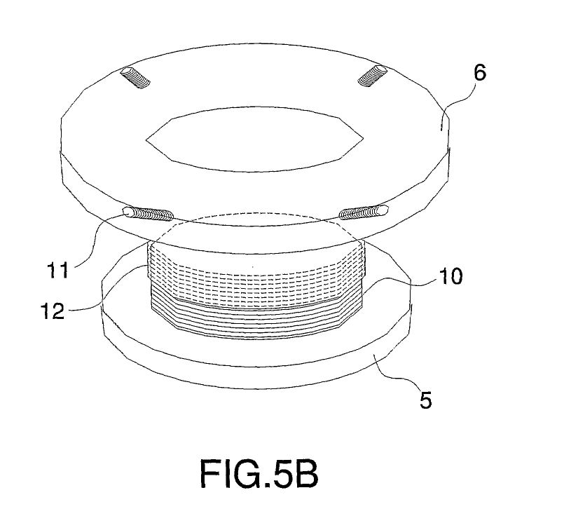

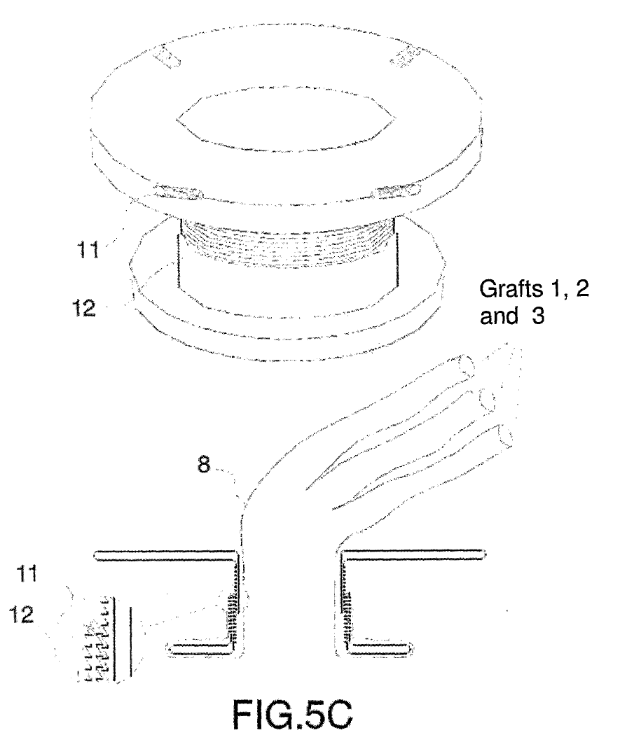

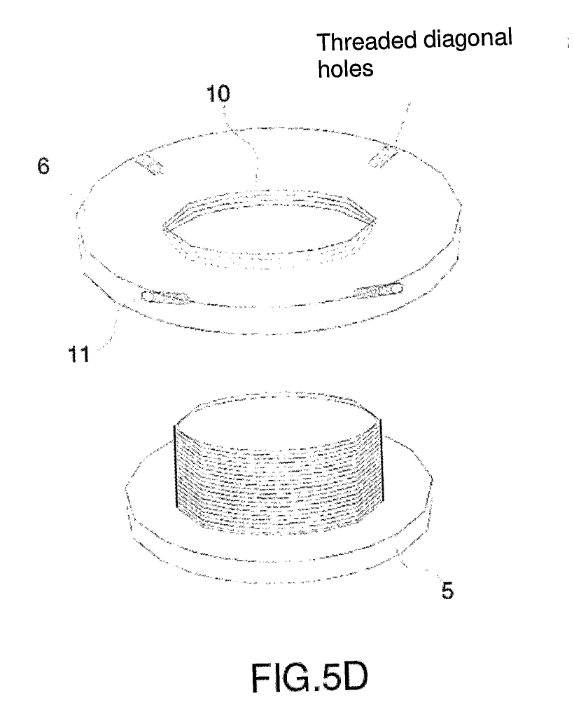

FIG. 5A differs from FIG. 3 in that it has small screw threads 10 inside the distal extremity of the tubular member of the external part 6 and, externally, the upper extremity of the tubular member of the internal part 5. These small screw threads 10 have the purpose of facilitating the use of the prosthesis that would be mobilized as if it were a single piece, guaranteeing at the same time the fixation of the parts between themselves, independently from the threads that will be passed through its flanges. The technique of its use is similar to those described above for FIGS. 3 and 4.

FIG. 5B represents a prosthesis with two parts, the internal 5 and external 6 ones, double flange, internal and external, screw threads 10 throughout the extension of the internal faces of the tubular member of the external part (and transversal grooves on the external face 12), and of the external face of the tubular member of the internal part. Besides having at least one oblique hole 11 with screw thread in the thickness of the flange to which a threading stick will be fitted by threading one part to the other until their complete adjustment by sandwiching to the thickness of the organ wall, making sure that there will be no protuberance of the prosthesis-grafts set while avoiding at the same time interposition at the origin of the anastomosis of unwanted material: threads, wall fragments, atheromatosic plaques, etc. To thread the parts between them, after introducing the prosthesis into the organ lumen, it is necessary that the internal part 5 be fixed with the nippers especially developed for this purpose. The technique for its use is precisely the same as described above for the prostheses of FIGS. 3 and 4. FIG. 5C represents a two-piece prosthesis, the internal 5 and external 6 ones, differing from the previous ones by the fact that the tubular member of the external piece has a smaller diameter than that of the internal piece. Thus, the screw threads 10 are located externally to the tubular member of the external piece and internally to the tubular member of the internal piece, which also has external grooves 12 for fixing of the grafts. On the upper rim of the tubular member of internal piece 5 there is at least one oblique hole 11, with screw thread, where another threading stick, similar to the one mentioned above, will be threaded with the purpose of fixing the internal piece, already introduced, at the moment of the threading of the pieces between them. As was seen in the first drawing of FIG. 5B, the external flange also presents on is free rim, obliquely, holes 11 with screw thread, where also another threading stick will be fixed to. Of course, once the sticks have been used, they will be removed. It is to be noted that in this case the anastomotic trunk 8 will be fixed with external circular point to the external surface of the tubular member of the internal piece 5 and not to the external piece 6, as in the other prostheses of FIGS. 3 to 5B. The prosthesis of FIG. 5D is made up by two pieces, in which the external one 6 is also a flange with internal screw thread 10 and threaded holes 11 on its free rim. The internal part 5 with tubular member of a smaller external diameter has threads on its external surface and a flange with a diameter smaller than that of external piece 6. Fixation of the internal piece 5 for threading the pieces together after introduction of the prosthesis to the organ lumen is made with nippers especially developed for this purpose. It is clear that the grafts, after being everted, will be externally fixed to the tubular member of internal piece 5. The technique for its use is similar to the previous ones of FIGS. 5B and 5C.

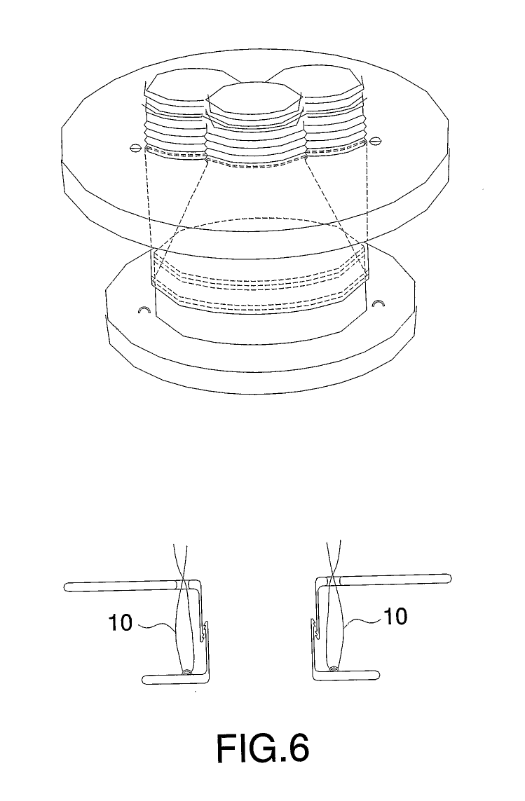

The prosthesis of FIG. 6 differs from the prosthesis of FIG. 4 only in that it has small screw threads 10 on its tubular members, internally to the external one and externally to the internal one. This, as was described, facilitates handling the prosthesis as if it were a single piece and guarantees their fixing together, independently of the threads that will do the sandwiching of the organ wall by approaching its pieces. It differs from the prosthesis of FIG. 5A only in that its external piece has at least one head (here, 3). Also the technique for its use is similar to that of FIGS. 3 to 5A.

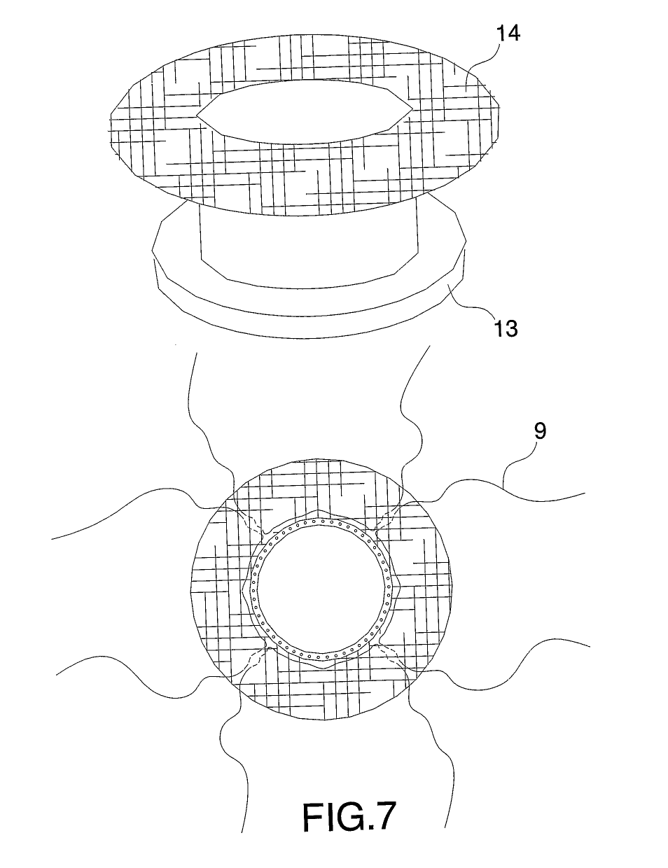

The prosthesis of FIG. 7 represents a prosthesis in a single piece with two flanges, one rigid and smaller internal one 13, and the other flexible and external one 14, made of biological or synthetic tissue and with a bigger diameter. In this external flange 14 holes are not necessary for the threads 9 may transfix it anywhere. Due to the fact that the flange is flexible and dispenses the need for holes, perhaps it is easier to use this prosthesis than those with rigid flanges with holes, if nothing else to apply extra stitches in case those initially applied break up at the moment they are tied down. The technique for its use is similar to the prostheses in FIGS. 1 and 2, already described, and may have other technical realizations.

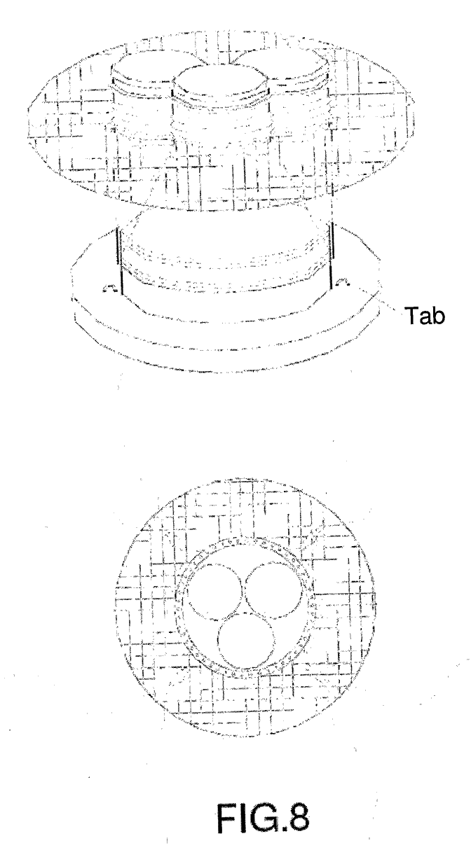

The prosthesis of FIG. 8 differs from those of FIG. 6 in that it also has a flexible external flange in biological tissue of any origin (either animal or vegetal, absorbable or unabsorbable) or synthetic tissue (Dacron, PTFE, Gor-Tex, silicone, etc.) similar to the prosthesis of FIG. 7. The technique for its application is similar to that of FIG. 6 already described.

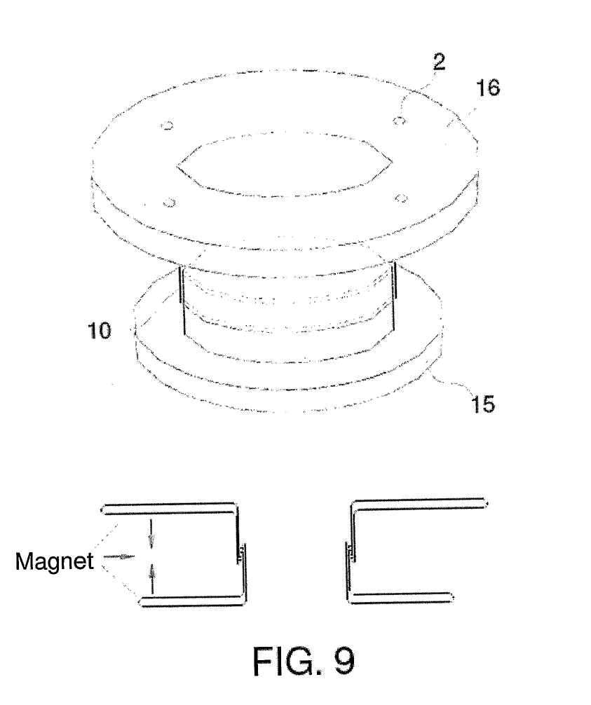

The prosthesis in FIG. 9 is made up by two parts, the internal 15 and external 16 ones. They are magnetized and so, they attract each other. They also have small screw threads 10 in their tubular members only to keep them united as if they were a single piece. After their introduction into the organ lumen, they automatically will make the perfect adjustment of their pieces onto the organ wall by sandwiching. If wanted, they also may be introduced in the middle of the suture in pocket into the organ surface to guarantee hemostasia and its fixation to the organ wall especially if it is friable, calcified or fibrosed (lacking elasticity).

Also at least two opposing threads with parallel legs may be applied through holes 2 on the upper flange, also to make sure that there will be hemostasia and fixing of the anastomotic set to the organ wall.

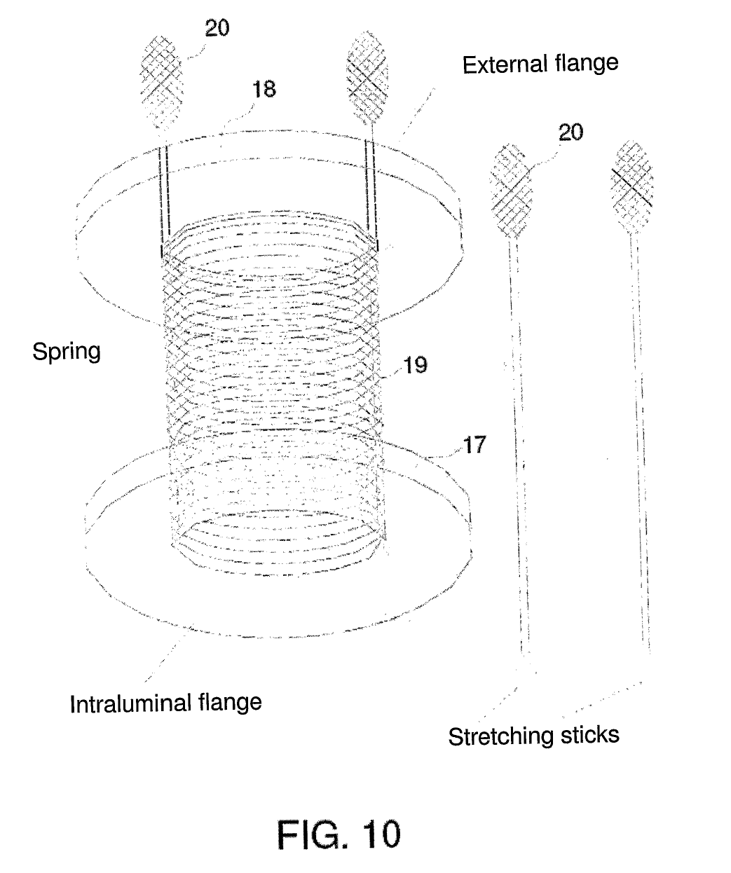

The prosthesis in FIG. 10 has a double flange 17,18 and a tubular member 19 replaced by a spring-type elastic device which, when at rest, keeps the two flanges together. By using the stretching sticks 20 that support themselves on the holes on the bases of the two flanges, the flanges are set apart and a prosthesis-grafts set or anastomotic trunk is assembled, and then may be fixed above the spring, after everted, with single stitches by removing the sticks 20, whereupon the sandwiching of the organ wall and the fitting of its thickness occur, avoiding protuberance of the set in its lumen. Here also fixing threads may be passed through the holes of the external flange, or the prosthesis may be introduced at the center of the suture in a pocked made on the surface of the organ, as described. It should be stressed again that for any prosthesis with internal flange to be covered without harm must have its natural caliber of the graft, if single, necessarily expanded on the extremity that will pass by the lumen of the prosthesis and will cover it by eversion. If grafts are multiple, making the anastomotic trunk is necessary for compatibility of the sum of diameters of the grafts with the external diameter of the internal flange so it may be covered by eversion. If the trunk or single graft has a diameter equal to the lumen of the prosthesis, there will be considerable difficulty for its eversion due to the larger diameter of the internal flange and, during attempts to make it, lacerations may occur on the intima layer of the grafts, a possibility which should absolutely not occur. If a graft with a diameter equal to the internal flange passes by the lumen of this prosthesis, it will be furrowed with many folds in its course through the prosthesis, which should not occur either. Thus, compatibility of the diameters of the grafts with the diameter of the internal flange is key to prevent that any anything of this sort occur. The trunk must have sufficient extension and diameter to cover all the intraluminal part of the prostheses. Preferably, the grafts must be individualized outside the lumen of the prosthesis and not in its interior on the level of the origin of the anastomosis. This makes sure that even if, for some reason, a compromising occurs at the origin of the anastomosis, with a much larger diameter than that of each individual graft, such as wall fragments, atheromatosic plaques, fibrosis, etc., no compromising will occur by stenosis or obstruction of the lumen of each graft, as they will be situated far from this spot. Surely, if this does occur, what is left from the lumen in the origin of the anastomosis will be enough to perfuse the organs to which they were directed, since the organs would show ischemic symptoms only if the compromising were over 70% of the original anastomotic area. This means that the use of large caliber, short prostheses whose intraluminal parts are covered by anastomotic trunks, making multiple anastomoses at one time with a single prosthesis without clamping and without suture is undoubtedly a big plus for the safety and longevity of the anastomotic procedures of any organ, especially vascular anastomoses in fine-wall, friable or calcified vessels. In the latter case there will be very little area mobilization for anastomosis, and it is possible to make as many as wanted at one time, besides the fact that the anastomotic set, considering its technical features, will surely reinforce the structure of the wall at the spot instead of weakening it, as is the case with conventional anastomoses.

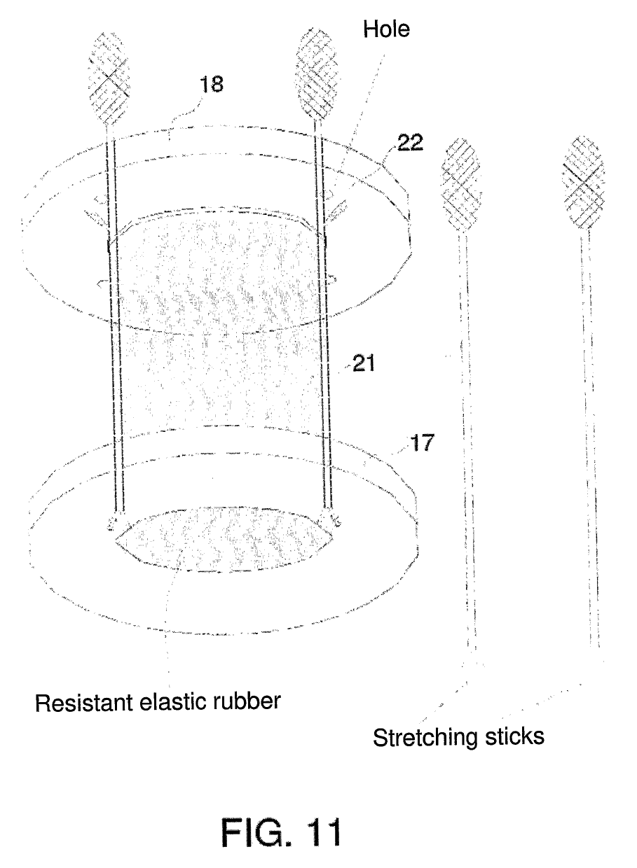

The prosthesis in FIG. 11 represents a prosthesis that is different from the one in FIG. 10 only due to its tubular member which, in this case, is made up by a resistant polymer 21, of an elastic and biocompatible rubber type that will work like the spring in FIG. 10. On the lower surface of its external flange, there is a small hole 22 to anchor the stem of the stretching stick. The technique and considerations are equal to those of FIG. 10.

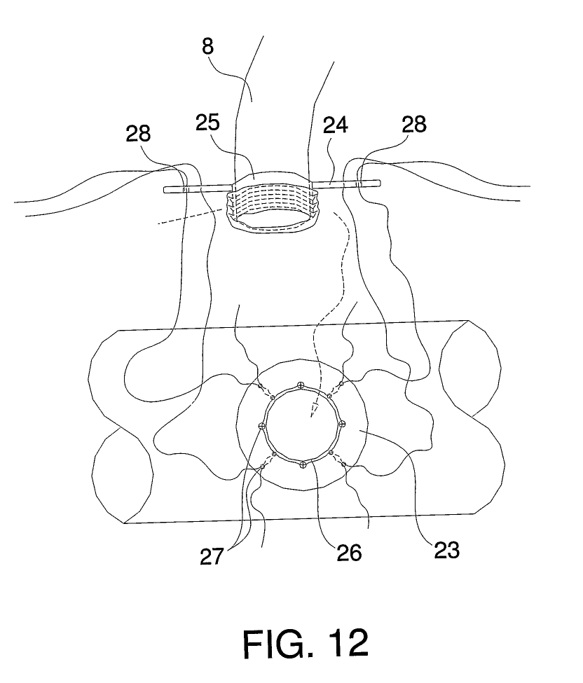

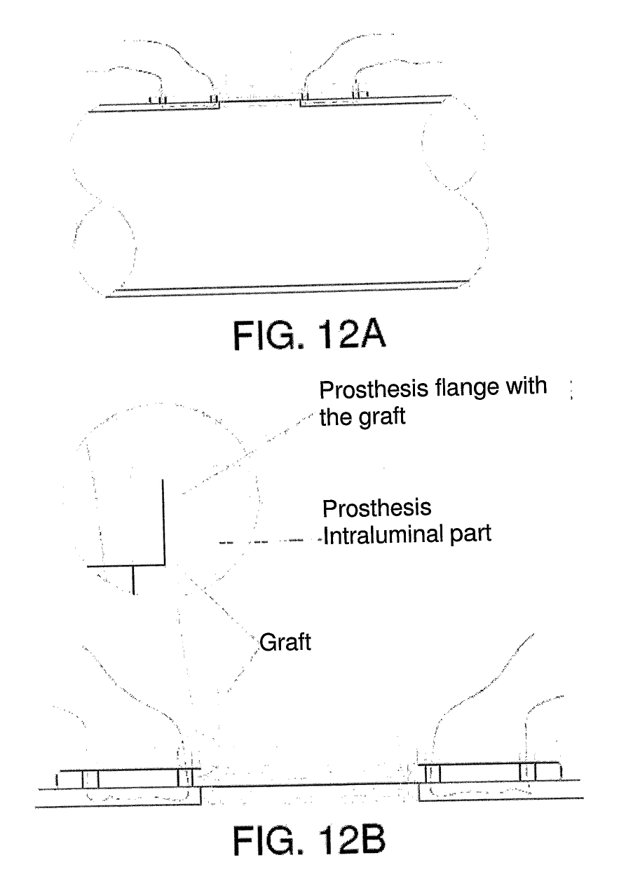

The prosthesis in FIG. 12 is made up by two parts, a second flange 23 and a first flange 24 from which a tubular member 25 orthogonally exits, being coated by the casing of the graft used. The second flange 23 has an internal diameter allowing the tubular member 25 of the first flange 24 to penetrate it tightly as represented in FIGS. 12A and 12B. Orthogonally to the internal rim of the second flange 23, there is a small upper fold 26 (detail in FIG. 12B), which will fit into the existing groove on the lower surface of the first flange 24 in order to make a perfect fitting easier between the parts and the hemostasia. Fold and groove may be inverted in their positions, with the fold staying on the first flange 24 and the groove on the second flange 23. Just the same, the second flange 23 has holes and double holes 27 through which the four stitches that will fix it to the external surface of the organ wall will pass. The first flange 24 with tubular member 25 coated by any kind of graft, preferably autologous graft, also has holes 28 coinciding with that of the second flange 23, as the same threads that served to fix the second flange 23 will pass from down up through these holes 28, facilitating the total fitting and fixing of the two parts. Thus, the second flange 23 is initially set on the organ wall and its threads pass through holes 28 of the first flange 24, whose tubular member 25 is already coated by the graft fixed to it. With a punch (cutting tool), the part of the organ wall inside the lumen of the second flange 23 is dried. Plugging is made with the finger. The prosthesis-graft set is introduced into this tight opening, and the threads are tied, thus ending the fixing of the two parts of the prosthesis. Since, obviously, the upper part has an extension of the intraluminal part equal to or discreetly larger than the thickness of the second flange 23, upon being introduced by passing by its lumen, it will be as if resting on the level of the upper surface of the organ wall and, in this case, instead of having a protuberance, the prosthesis-graft set will be above the internal level of the organ wall, as seen in FIGS. 12A and 12B. If made with large caliber anastomotic trunk, it will be with a big anastomosis area, still without clamping and any kind of foreign body, which will give much safety in hemostasia and longevity of these anastomoses. The second flange 23, when fixed to the organ wall, also fixes between itself the layers of the organ wall as well as the structures internally adhered to it, such as calcifications, atheromatosic plaques, etc., preventing them from detaching during anastomosis and embolizing to any place.

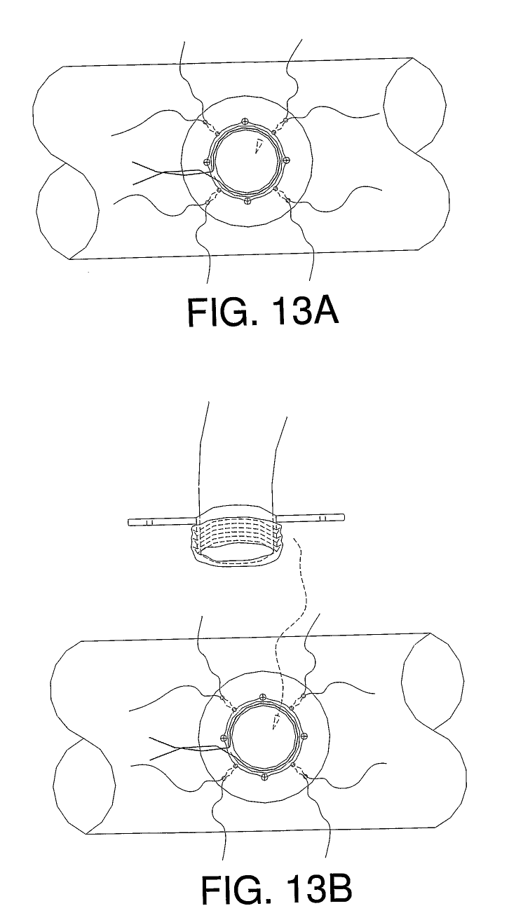

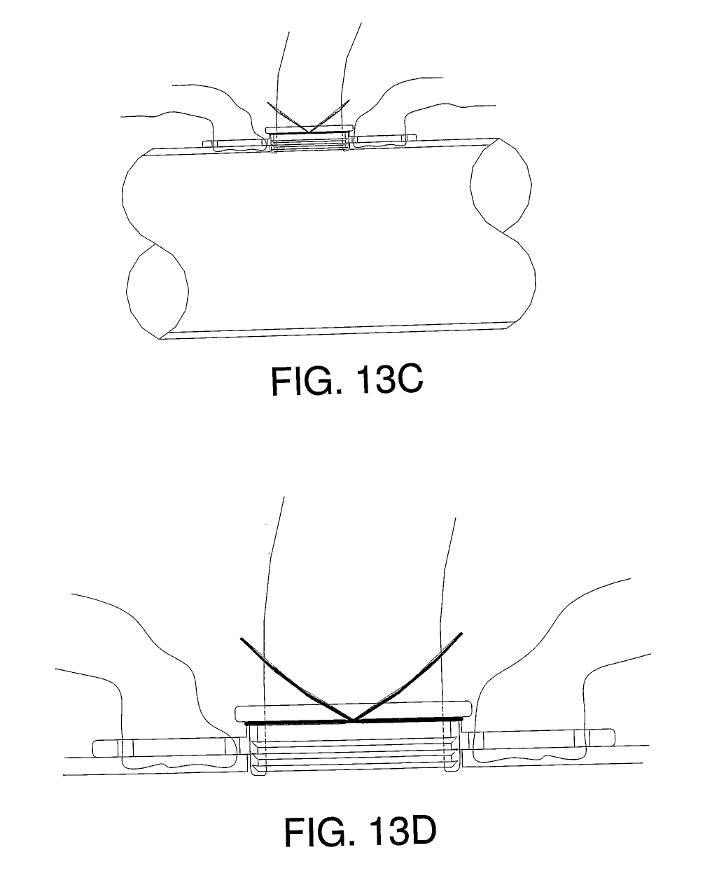

FIG. 13A presents an anastomotic set that differs from that of FIG. 12, just because the second flange 23 has circumferentially to its lumen a suture type in pocket fixed to it by any safe method (such as, for instance, suture, glue, etc.), tunneled tissue, single or double, with opposing exits, with very resistant thread inside it. When the prosthesis-graft set is introduced in its lumen and the lumen of the organ, this thread or threads are driven and the tissue pocket will involve the set facilitating its fixing and perfect hemostasia in the anastomosis. This is illustrated in FIGS. 13A, 13B and, in the detail, in 13C and 13D.

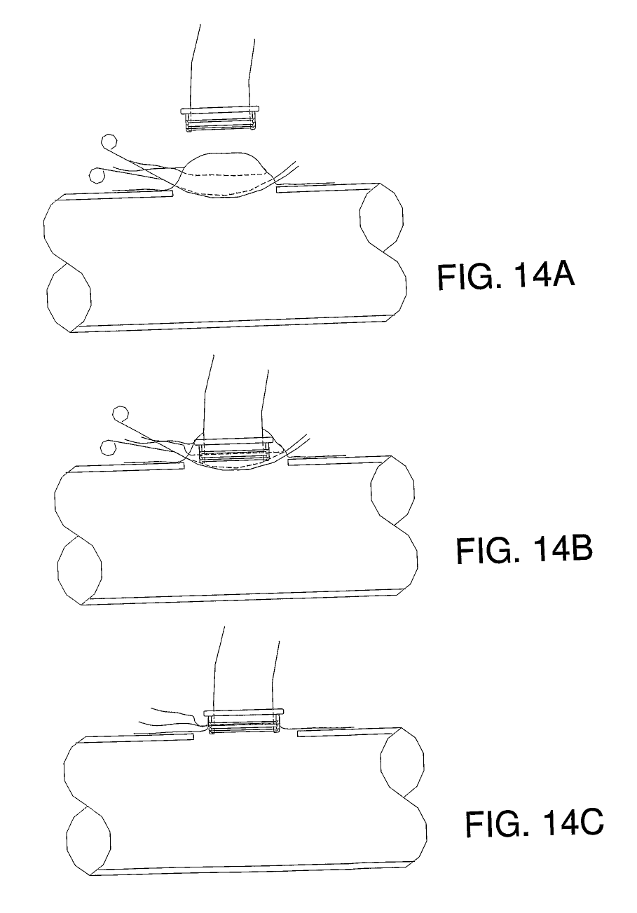

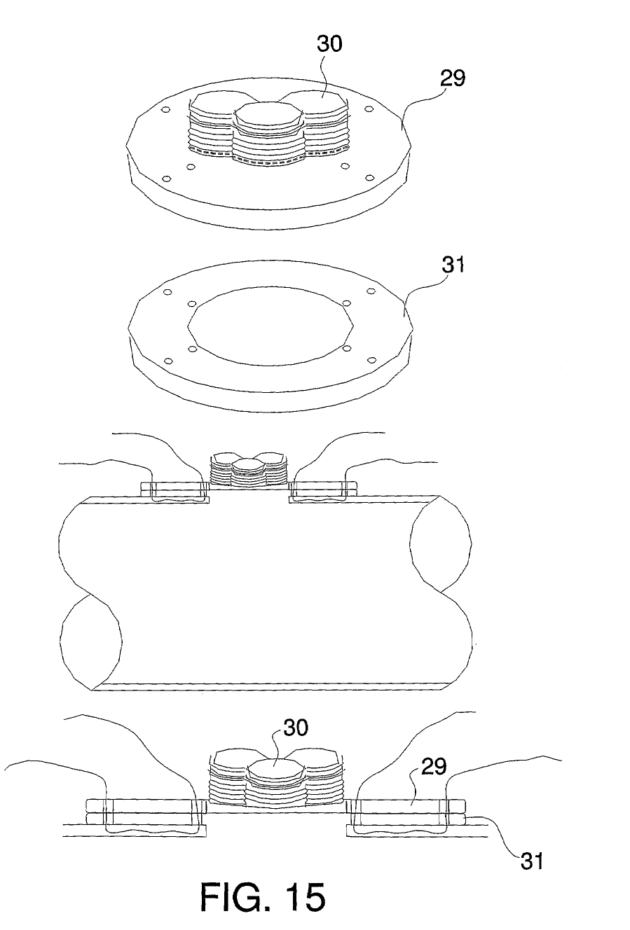

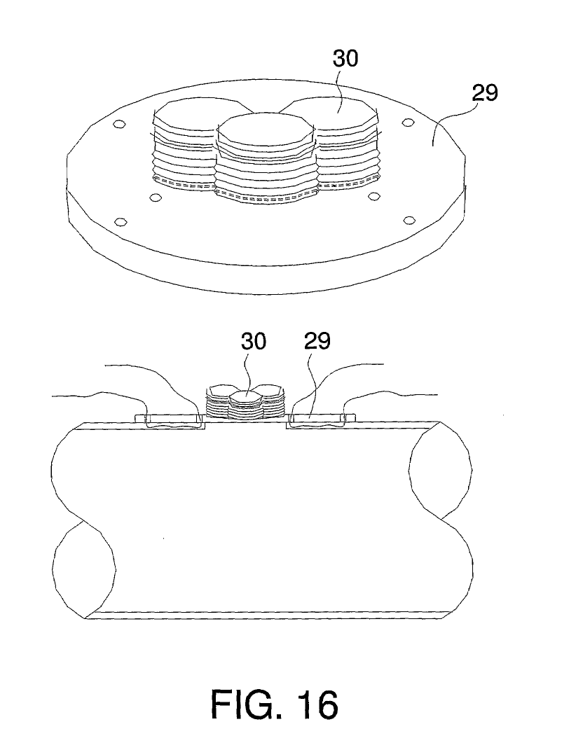

The anastomotic set represented in Figures from 14A to 14C replaces the second flange in FIG. 12 by any biological or synthetic tissue, preferably autologous (pericardium), which will be doubly sutured at once onto the organ wall (the stitch passes in the graft from up down on the wall, either transfixing it or not, and peripherally returns to the graft from down up and then it is tied) in such a way that it will be overbound, that is, it will form a small pocket on the organ wall. A pocket suture is made on its base, either single or double and opposed externally to this graft, more or less in the diameter of the anastomotic set. Incise it viewing the organ wall that will then be excised with the circular punch with a diameter equal to that of the anastomotic set or larger. The anastomotic set is introduced through the incision of the graft and the pocket threads are driven, being fixed on the anastomotic set so as the anastomosis is ended. Also here, there will never be a protuberance of the set in the lumen of the organ, but rather, since its base is flexible (graft sutured to the organ wall), it will be “thrown out” if the organ presents pressure, such as a vessel. At last, FIG. 15 presents an anastomotic set that differs from those described in FIGS. 12 to 14C as they have two flanges without intraluminal tubular members. The upper flange 29 has a triple head 30 whose grafts will cover them individually and externally, being fixed to them with an external circular point or any other safe method. This upper flange 29 will have a fold or groove on its lower surface that will perfectly fit into the groove or fold located on the upper surface of the lower flange 31, which will guarantee fixing the set and a perfect hemostasia, as seen in the detail of FIG. 12B. The number of grooves and folds may be multiplied provided that they are tightly fitted. The lower flange 31, once fixed to the organ wall, insulating the small segment of this wall and after drying of this segment by circular punch in a previously set diameter as equal to the lumen of the lower flange, will have its lumen provisionally occluded, if it is a vessel, by the finger of the surgeon or his/her assistant.

Finally, FIG. 16 represents a variation on the prosthesis of FIG. 15, in which only its flange above 29.30 is used without using the lower flange 31. In this case, the upper flange 29.30 will be directly linked to the organ wall.

This way, it implies that it must be understood that the prosthesis with multiple flanges for components and their parts described above is only one of the modalities and examples of situations that might occur, as the real scope of the object of the invention is defined in the claims.

1. Prosthesis for anastomosis comprising a first tubular member, a lumen and a first flange (1) extending from a lateral wall of the tubular member; the flange having a plurality of through openings (2) distributed throughout its surface; and a second flange (3) opposite the first flange (1), extending from a lateral wall of a second tubular member.

2. Prosthesis for anastomosis, according to claim 1, wherein the first and second tubular bodies form a single part.

3. Prosthesis for anastomosis, according to claim 2, wherein the tubular body in a single part may be made of various materials, including a same material of the flange, spring, or elastic rubber.

4. Prosthesis for anastomosis, according to claim 3, wherein the tubular bodies of flexible material are stretched by stretching sticks (20).

5. Prosthesis for anastomosis, according to claim 1, wherein the first and second tubular bodies are connected by screw thread (10).

6. Prosthesis for anastomosis, according to claim 1, wherein the first and second tubular bodies are connected by compression exercised by sandwiching of the wall of the anastomosed tissue.

7. Prosthesis for anastomosis, according to claim 1, wherein the first and second tubular bodies are connected by magnetic attraction.

8. Prosthesis for anastomosis comprising a tubular member (25), a lumen and a first flange (24) extending from the lateral wall of the tubular member (25); the flange (24) having several through openings (28) distributed throughout its surface; and a second flange (23), independent from the first flange (24) without an intraluminal part.

9. Prosthesis for anastomosis, according to claim 8, wherein the first (24) and second (23) flanges have the same diameter and contiguous through holes (27.28).

10. Prosthesis for anastomosis, according to claim 8, wherein the second flange (23) has a fixing mechanism by pocket constriction on its internal rim.

11. Prosthesis for anastomosis, according to claim 8, wherein the second flange (23) is a synthetic or biological graft.

12. Prosthesis for anastomosis comprising: two flanges (29, 31) of equal diameter, lacking tubular members, that couple together and are fixed by through points (27, 28) and grooves and folds that fit tightly together.

13. Prosthesis for anastomosis, according to claim 12, wherein it includes only one flange (29) with heads (30) without a tubular member.
